(19) United States
(12) Patent Application Publication
(10) Pub. No.: US 2010/0278394 A1
(43) Pub. Date: Nov. 4, 2010

(54) Apparatus for Iris Capture

(76) Inventors: Daniel H. Raguin, Acton, MA (US); George W. McClurg, Jensen Beach, FL (US); John F. Carver, Palm City, FL (US); Adam Mark Will, Fort Mill, SC (US); Christopher Robert Fulmer, Jupiter, FL (US); Dean J. Fedele, Jupiter, FL (US); Andreas Bauermeister, Thuringia (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/607,677

(22) Filed: Oct. 28, 2009

Related U.S. Application Data
(60) Provisional application No. 61/109,361, filed on Oct. 29, 2008.

(51) Int. Cl. G06K 9/00; A61B 3/14; A61B 3/15

(52) U.S. Cl. 382/117; 351/206; 351/208; 382/124

(57) ABSTRACT

An apparatus comprising an illumination source and an imaging sensor is provided to measure an iris for biometric identification purposes. The illumination source is configured to illuminate an eye to produce reflected light. The imaging sensor is configured to receive the reflected light. An optical path is formed for the reflected light from the eye to the imaging sensor.

### CROSS REFERENCE TO RELATED APPLICATIONS

The present application claims the benefit under 35 U.S.C. §119(e) to U.S. Provisional Patent Application No. 61/109,361, entitled “Apparatus for Iris Capture”, and filed on Oct. 29, 2008, which is incorporated by reference herein in its entirety.

### BACKGROUND

1. Field of the Invention

The present invention relates generally to the field of biometrics, and in particular to iris scanning devices used to identify persons or other living beings based on biometrics associated with the eye.

2. Related Art

Biometrics is a science involving the analysis of biological characteristics. Biometric imaging captures a measurable characteristic of a human being or other living organism, typically a mammal, for identity purposes. Eye scanners are biometric imaging systems for acquiring images of the human eye or the eye of other mammals for identity purposes. One common type of eye scanner is the iris scanner, which relies on the distinctive patterns of the human iris to distinguish one individual from another.

The use of iris images for the identification of individuals has gained acceptance in today's society as a reliable means of distinguishing between two people and to confirm an individual's identity. Devices that capture iris information have been documented for at least the past 20 years. Through the capture of iris images, subjects can be enrolled into biometric databases and subsequently identified for both government and civil applications. Iris images provide increased accuracy over the use of printed identification cards, which are typically only loosely tied to an individual via a facial photograph.

Several challenges exist in modern-day iris scanner technology. Optical elements of eye scanners need to be correctly aimed at the eye or eyes of a user, and further the optical elements need to be focused correctly on the physiological features of interest (i.e., the irises of the eyes) to obtain quality images of these features. Additional requirements for effective scanning include ensuring that the eyes are shielded from ambient light, which may interfere with effective image capture, while at the same time aligning the eyes of the user with illumination from the scanner, which is intended to illuminate the eyes for purposes of image capture.

Ensuring correct focus of an eye scanner on the physiological features of interest, and further insuring correct alignment, may involve correct translational positioning of optical components relative to an eye along a line of sight from the eye. It may further involve ensuring that the optical components are at a correct height relative to the eye or eyes of a user. As opposed to livescan fingerprint devices, in which a finger or fingers are in contact with a platen, and therefore a fixed distance from the imaging optics, a subject's iris is generally not at a fixed distance with respect to the imaging optics.

For example, one approach to a sensing system may employ variable optical elements, e.g., a mechanical focusing mechanism, which may in turn entail gears, rails, springs, internal hydraulics, or similar elements. Such mechanical focusing mechanisms based on translational movement may move or otherwise adjust lenses or other optics to move closer or further from an eye along the line of sight of the eye to bring features of interest into an in-focus condition. However, such focusing mechanisms introduce substantial mechanical complexity, along with a requirement that a determination be made via some means or mechanism to ascertain when the desired physiologic features of the eye are actually in focus. Such translational movement of optics further-from and closer-to the eye may also be cumbersome and undesirable for users.

Another possible approach of focusing and alignment is to allow the person being measured to move their head in relation to the scanning mechanism, until the eye is in the proper position for proper focus. However, this approach may require a dynamic determination to be made as to when the person's eyes are at the proper distance from the scanning device, or at the proper height or correct angle relative to the scanner device. A further requirement is to provide visual indicators that signal to a person that he or she should move the head forward or backwards or in other directions, or keep the head at the current location.

Again, design complexity ensues. Moreover, such a system may also pose a challenge for some users who have difficulty following the visual cues that are intended to guide the position of their eyes or head. Such movement of a person closer to or further from an eye scanner may be awkward in many applications such as remote field use in hostile environments.

Moreover, these two challenges of focus and alignment must be addressed in the context of a modern-day iris scanner. Today, these devices consist of an illumination system, an imaging system, and a sensor. The illumination system generally comprises of an array of near infrared light emitting diodes (LEDs) that may be collimated or otherwise directed towards the location where the system expects to view the subject's face. As specified by the ANSI (American National Standards Institute) INCITS (International Committee for Information Technology Standards) 379-2004 (Iris Image Interchange Format) specifications document, the wavelength range of the illumination system is preferentially between about 700 nm and 900 nm. The imaging system generally comprises of an array of refractive lenses configured such that at a set distance L they are capable of projecting a high-quality image onto a high-resolution image sensor. According to ANSI INCITS 379-2004, to satisfy a high image quality, an MTF (Modulation Transfer Function) of greater than 60% at 4 lp/mm (line-pairs per millimeter) at the iris and an electronic resolution of greater than 16.7 pixels/mm are required. The sensor for the iris capture device is generally a two-dimensional CMOS detector element.

Although imaging an object at the required electronic resolution is straight-forward, in order to achieve the required optical resolution, high-quality optics must be procured and the optical system must be in focus for a given optical system to iris distance. As noted above, a subject's iris is generally not a fixed distance with respect to the imaging optics. An iris capture device can employ a wide field-of-view (FOV) camera and separately a narrow FOV camera. The wide FOV camera is used to locate a subject's face and through triangulation guide the narrow FOV camera in order to acquire a sharp focus iris image. Alternatively, a range finder device (e.g., acoustic or infrared) can be used in order to determine the distance the subject is from the iris capture device and to capture the image when the subject is in focus.

As noted above, a third issue associated with the ability to capture high-quality images of the iris is ambient light. Since the surface of the eye is smooth with a glossy appearance, the eye reflects ambient light, and the reflected ambient light generally erodes the image quality of the iris that is being captured. When capturing the iris of a subject that is indoors, one can reduce the effects of ambient light by having an illumination source (typically near infrared) that is significantly brighter than the indoor ambient light, while still maintaining eye safety. However, when capturing an image of a subject's iris outdoors, sunlight can easily be several orders of magnitude brighter than indoor lighting and, in certain applications (such as military or border patrol operations), one does not necessarily have the option of seeking shade in order to capture a subject's iris. All the aforementioned focus mechanisms that rely upon the subject not being in contact with the iris capture device have issues in terms of movement of the subject relative to the camera and ambient light.

### SUMMARY

What is needed is a mechanically and electrically simple means to ensure that a person's head, and in particular a person's eyes, are properly positioned in relation to an optical sensor in terms of distance, and other related location vectors, in order to ensure proper focus by sensor optics on the physiologic features of a person's eyes, with minimal scanner system complexity. What may also be needed is a system which aligns the eyes of the user with scanner-generated lighting, while shielding the eyes from unwanted ambient lighting. What may also be needed is a system and method which is simple, whose usage is straightforward for a typical or average user, and which is convenient for the user whose eyes are to be imaged via the optical scanning system.

Embodiments of the present invention describe an apparatus and method of capturing an image of at least a portion of an eye or eyes, and more specifically an image of at least a portion of an iris or irises.

In one embodiment of the present invention, there is provided an apparatus comprising an illumination source and an imaging sensor. The illumination source is configured to illuminate an eye to produce reflected light. The imaging sensor is configured to receive the reflected light to measure an iris for biometric identification purposes. An optical path is formed for the reflected light from the eye to the imaging sensor.

Further embodiments, features, and advantages of the present invention, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the pertinent art to make and use the invention. In the drawings, like reference numbers indicate identical or functionally similar elements. Further, and except where specifically noted otherwise, the drawing in which an element first appears is typically indicated by the leftmost digit(s) in the corresponding reference number (e.g., an element numbered 302 first appears in FIG. 3).

FIG. 1A is an illustration of a first view of an exemplary iris scanner in its closed configuration, according to an embodiment of the present invention.

FIG. 1B is an illustration of a first view of an exemplary iris scanner in its open condition, according to an embodiment of the present invention.

FIG. 1C is an illustration of an exemplary iris scanner in use with a subject, according to an embodiment of the present invention.

FIG. 2 is a cross-sectional view of the iris scanner, according to an embodiment of the present invention.

FIGS. 3A and 3B illustrate visible alignment aids for an iris scanner, according to an embodiment of the present invention.

FIG. 4 illustrates a solar radiation spectrum in a ultraviolet, visible and infrared wavelength ranges at sea level.

FIGS. 5A, 5B, 5C, and 5D depict simplified illustrations of optics of iris scanners, in accordance with various embodiments of the present invention.

FIG. 6 depicts a top perspective view of an embodiment of a portable biometric device, e.g., in a closed non-operating position.

FIG. 7 depicts perspective view of device in an open position.

FIGS. 8 and 9 illustrate an optical system located within or associated with a device, according to one embodiment of the present invention.

FIGS. 10A, 10B, 10C, and 10D show various views, e.g., exploded, collapsed, and extended, of an iris scanner, according to another embodiment of the present invention.

FIGS. 11A, 11B, 12A, and 12B depict mechanisms by which a distance between an iris of a subject and an iris scanning device is maintained, according to additional embodiments of the present invention.

The features, objects, and advantages of the present invention will become more apparent from the detailed description set forth below when taken in conjunction with the drawings in which like reference characters identify corresponding elements throughout.

### DETAILED DESCRIPTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

The embodiment(s) described, and references in the specification to “one embodiment”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Embodiments of the invention may be implemented in hardware, firmware, software, or any combination thereof. Embodiments of the invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

In one or more embodiments, the apparatus of the present invention, herein referred to as an iris capture device, incorporates a detector (e.g., a camera) comprising an imaging system and a sensor. The imaging system comprises one or a plurality of optical elements. The sensor is designed to capture an iris image of suitable quality and resolution to achieve the level of identification required for a particular application. Additionally, the iris capture device incorporates an illumination source that comprises at least one visible light source, at least one infrared source, or a combination thereof.

In one embodiment, one or more portions of the iris capture device open. In the closed position, the optics are protected from the environment, but in the open position the imaging systems of the device can capture the biometric information from subject. For example, U.S. patent application Ser. No. 11/868,403 to Carver et al., which is incorporated herein by reference in its entirety, discloses an iris capture device that has a rotating visor that sets the device-to-iris distance in addition to allowing for ambient light shielding.

In an embodiment of the present invention, no rotating visor is necessary. Instead, the device opens and creates a contact surface, such that one or more portions of the subject's face are in contact with the iris capture device. By touching the subject's face, the imaging system to iris distance is set, thereby alleviating the need for an alternative focus mechanism. Alternatively, or additionally, the portions of the iris capture device that open up can create an ambient light shield in order to suppress spurious reflections off of the subject's eye that would compromise the fidelity of the captured iris image. The ambient light shield can shield the subject's iris from ambient light from all directions (e.g., top, bottom, and sides).

In another embodiment, the iris capture device does not open, but instead has a window in the housing of the device that allows the imaging system to see a subject's eyes. The mechanics of the iris capture device housing are designed so that they can contact one or more portions of the subject's face directly. The imaging system is designed such that the subject's iris will be in focus. In one example, the housing itself allows for the ambient light shielding required in order to take a high-quality image.

In a further embodiment, the illumination source of the image capture device emits a beam of radiation at one or a plurality of wavelengths that corresponds to low intensity levels of the ambient light. For the case of the ambient light being sunlight, wavelengths include, by way of example, about 760.5 nm (corresponding to an O₂ absorption band) and the H₂O absorption bands of about 823 nm, 1100 nm, 1400 nm, and 1900 nm.

Typically, iris capture devices may not utilize wavelengths above about 900 nm as the sensitivity of low-cost silicon sensors (e.g., CMOS) falls rapidly above these wavelengths. The devices may utilize one or a plurality of the specified wavelengths below about 900 nm, but the devices do not capitalize upon the water and oxygen absorption bands in the solar spectrum since the illumination sources and spectral filters incorporated are broadband. In general, iris capture devices employ light emitting diodes (LEDs) that have spectral bandwidths greater than about 30 nm which, by way of example, is greater than about 10× wider than the spectral hole at about 760.5 nm or 823 nm. The spectral filters the devices incorporate can be hot windows that pass infrared above about 700 nm and reflect wavelength below this.

In other embodiments of the present invention, the use of illumination sources, spectral filters, or a combination of the two is specified to allow the suppression of ambient light, specifically solar radiation for the purposes of obtaining a high-quality image of the iris. For the absorption bands in the solar spectrum that are narrow (e.g., an about 3 nm hole centered at about 760.5 nm), the optical system can be designed, such that at a given plane the range of angles are minimized. Minimizing the range of angles may be important because to minimize an amount of angle of incidence induced center wavelength shift of a narrowband spectral filter. In one embodiment, the optical system range of angles are minimized by designing an optical system that is telecentric in image space and the required narrowband spectral filter is placed in image space.

In a still further embodiment, a visible light source is incorporated into the iris capture device for the purpose of guiding the direction at which the subject looks. The light source may, by way of example, be a fluorescent light, an arc lamp, an LED or a combination thereof. In one example, the light source is composed of one or more LEDs due to the LEDs' compactness and energy efficiency.

The visible light source may be combined with an optical system to form a subject alignment system, such that a virtual image appears at a distance sufficiently large that the subject's eyes can focus on it. In one embodiment, a target, which by way of example may depict a grid, crosshair, company logo or text, is projected to appear to be at a large distance from the subject's eyes. In another embodiment, a phase plate is integrated into the subject alignment system. The phase plate is designed such that the far-field diffractive pattern depicts an object, which, by way of example, may be a grid, crosshair, company logo or text. By illuminating the phase plate with approximately collimated light, the subject looking at the light transmitted by the phase plate will naturally accommodate their eyes for far-field imaging which will result in the far-field diffractive pattern of the phase plate illuminating their retina, thereby creating the sense that they are looking at the image of an object in the far-field, and therefore more relaxing for the subject's eyes.

Exemplary embodiments are described in terms of exemplary iris scanners, which may be used for identifying persons based on features of a person's iris. However, the embodiments of the present invention may equally well be employed in the context of other eye scanning devices which may scan, for example and without limitation, the human retina or the pattern of blood vessels of the choroid (which may be visible through the sclera).

Embodiments illustrated herein may be portable eye scanners, and may further be handheld eye scanners, but the present invention is not limited to such devices. It will be apparent to persons skilled in the relevant arts that the present system and method may apply equally to non-portable eye scanners and to eye scanners which are held in place in relation to a person's head and/or eyes by means other than being held in the person's hands.

For brevity, this document sometimes uses the singular term “eye”, or the plural term “eyes”, where it may be understood that either the singular term “eye”, the plural term “eyes”, or both may be applicable, depending on particular configurations of particular embodiments of the present invention.

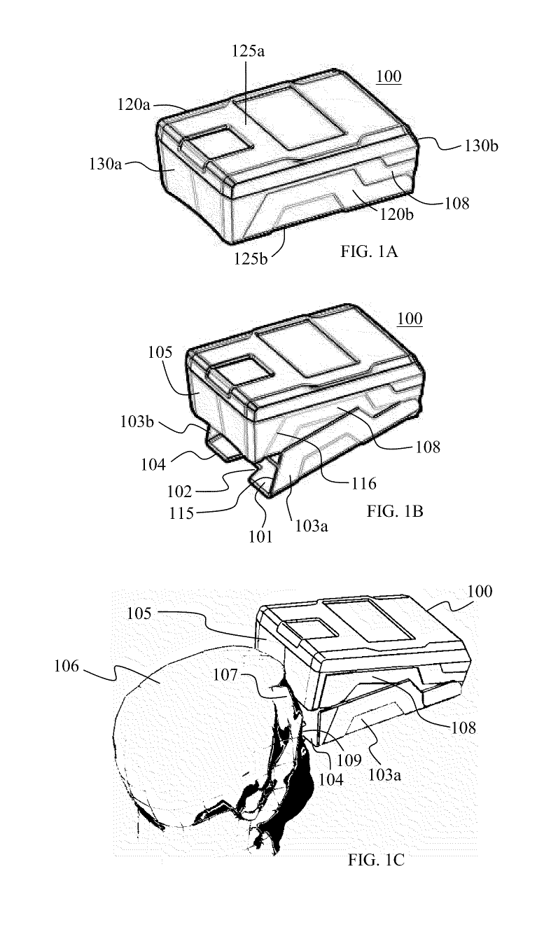

FIGS. 1A, 1B and 1C depict an iris capture device 100, according to one or more embodiments of the present invention. For example, iris capture device 100 may address two of the fundamental issues facing iris capture devices, namely the setting of iris to optical system distance and ambient light rejection. Though the following teachings will refer to the eye, iris, forehead and other facial features of a human head, it is intended that the disclosed invention may be applied to the capture of non-human eyes.

In one example, device 100 comprises a housing 108, a bottom plate 101, an optional recessed feature 102, e.g., configured to accommodate a nose of a subject, one or more side shields 103a and 103b, a bottom cover 104, and a housing front 105. Also depicted in FIGS. 1A, 1B, and 1C are a head 106 of a subject, a brow or forehead 107 of the subject, and an eye or eyes 109 of the subject (although not clearly shown). It is to be appreciated that all references to perspective (e.g., bottom, top, side, front, etc.) are with respect to as shown in the Figures, but not by limitation. In one example, housing front 105 can be curved in order to better match the curvature of the subject's brow or forehead 107.

As depicted in FIG. 1A, device 100 is illustrated in a closed position. In FIG. 1B an open position of device 100 is illustrated, where a bottom cover 104 of the device separates from the rest of housing 108 and allows an optical system (not shown) within device 100 to be able to view an outside biometric feature and specifically capture iris images. As illustrated in FIG. 1B, bottom cover 104 contains features that aid in suppression of ambient light and for the setting of iris distance. Bottom cover 104 incorporates side shields 103a and 103b, as well as bottom plate 101, for the purposes of shielding eyes 109 of the subject from ambient light coming from the left or right sides of device 100, as well as from the ground. A stationary portion of housing 108 can shield eyes 109 of the subject from ambient light coming from above.

Additionally, or alternatively, an iris capture device comprising a separating portion that provides for an ambient light shielding tunnel and has a moving top and stationary bottom portion of the device may be implemented. Also additionally, or alternatively, a device comprising sides that open and swivel out to create a similar ambient light shielding tunnel may be engineered.

FIG. 1C illustrates an exemplary operation of device 100. For example, bottom cover 104 is flipped down and device 100 is positioned in front of head 106, such that housing front 105 makes contact with brow or forehead 107. In one example, the nose may be optionally aligned or supported in recess 102 of bottom cover 104. This alignment allows eyes 109 to be viewed by the optical system contained within housing 108.

In the embodiment shown, a mechanical stop arrangement of the present invention is designed to fix a distance between a portion of the subject's face and the optical system of the image capture device. By way of example, the portion of the subject's face may be the brow, cheekbones, or nose. Contouring can provide reproducible contact with the subject's face such that the subject's eyes are the appropriate distance from the iris scanning device 100. The overarching contouring requirement is that it defines a plane on the head of the subject that is perpendicular or substantially perpendicular to the optical axis of the iris illumination and imaging system. One skilled in the art will appreciate that a wide variety of approaches to the contouring requirement will meet this objective and are within the scope of embodiments of the present invention. Specifically, contouring can be designed to provide three-point contact, a line contact, or two points and a line contact with the subject's face. The two points and a line approach define contact via a line across the forehead of the subject, with the two points being, e.g., each cheek of the subject's face.

With any means of fixing the distance between subject's eyes and the scanning device, there will be a small variation in the distance between the iris and imaging optics that will be present due to subject-to-subject variations in the recess distance of the eyes relative to the facial feature(s). In one example, the object distance variation is substantially eliminated by designing an optical system that has the required depth of focus (for example, by stopping down, i.e., reducing the f-number of the optical system).

In one embodiment of the present invention, device 100 is a portable device. As such, it is desirable that moveable bottom cover 104 illustrated in FIG. 1B seals the internal optics and mechanics from the outside environment when in the closed position. By way of example, side shields 103a and 103b and bottom plate 101 of bottom cover 104 may slide into feature 116 or gasketing (not shown) of housing 108 to protect the internal mechanics from the environment in the closed position. By way of example, feature 116 of housing 108 may be a slot designed to accept edge 115 of side shield 103a, and thereby help seal the internal mechanics and optics of apparatus 100 from the external environment. A similar slot arrangement can be designed for the opposing side shield 103b. In the open position, a window (not shown) may optionally be used to protect the internal optics from contamination. Such a window can be made of a substantially transparent material, with respect to the wavelengths being used, which by way of example could be glass or plastic such as borosilicate glass, polycarbonate or acrylic. For applications requiring severe abrasion resistance, a window composed of sapphire may be considered or alternately a glass or plastic window with a hard coating added.

In one embodiment of the present system and method, scanner housing 108 may be approximately a rectangular parallelpiped in shape, with three pairs of facing, approximately planar surfaces or walls 120a, 120b, 125a, 125b, 130a, and 130b, defining a substantially closed scanner housing 108. For any given pair of facing planar surfaces, e.g., 120a and 120b, a first planar surface 120a of the pair may be substantially or approximately parallel to a second approximately planar surface 120b of the same given pair. Each pair of the three pairs of facing approximately planar surfaces may be substantially or approximately orthogonal to each of the other two pairs of facing approximately planar surfaces.

In alternative embodiments of the present system and method, scanner housing 108 may have other shapes including, for example and without limitation, approximately ellipsoid, approximately triangular, approximately trapezoidal, approximately cylindrical, and approximately pyramidal. As mentioned earlier, face 130a may be curved in a manner as to mate to the brow or forehead of a subject whose iris(es) is being imaged. Other shapes are possible as well within the scope and spirit of the present system and method. For example, shapes also include binocular, monocular, and wrap-around visor shapes. While scanner housing 108 is discussed and illustrated throughout this document as having an approximately cuboid shape for purposes of presenting embodiments of the present system and method, scanner housing 108 is not limited to an approximately rectangular block.

In various examples, scanner housing 108 may be composed of a material or combination of materials, such as plastic, various metals or metal alloys, various polymers, or various composite substances well known in the art. Such materials may be rigid enough to provide the necessary structural sturdiness for scanner housing 108 to operate properly (for example, to provide support for internal structural and functional components, or to establish a substantially fixed distance between the forehead of a user and the optical elements of the scanner); yet such materials may also have sufficient ability to bend or flex, that is, may have a sufficient elasticity, to support the operation of moving elements.

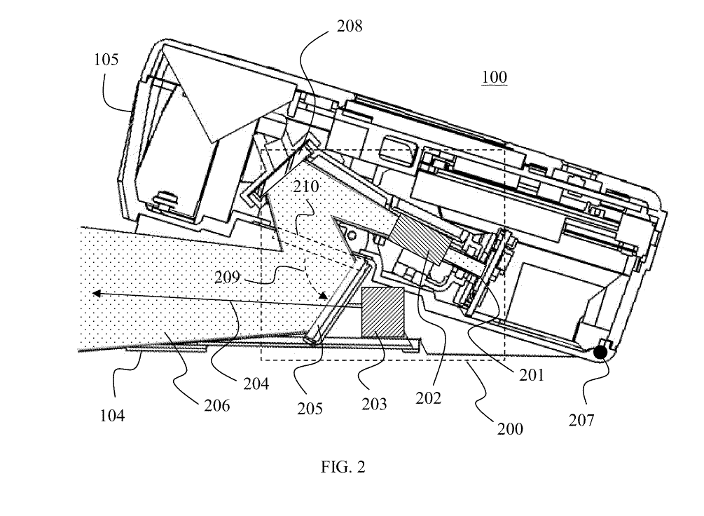

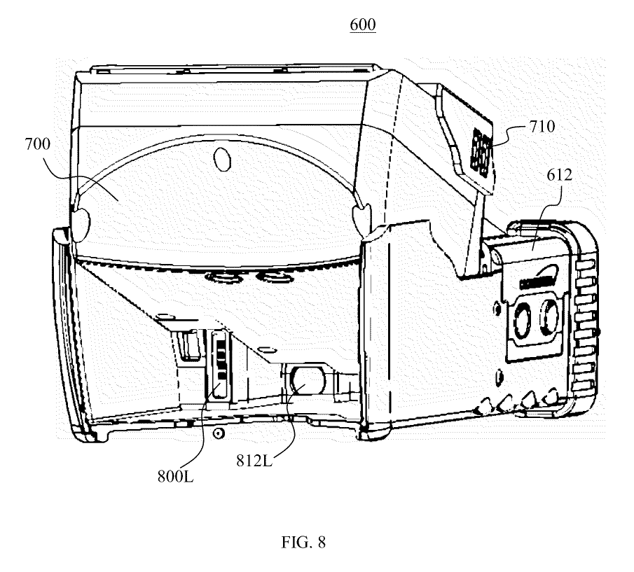

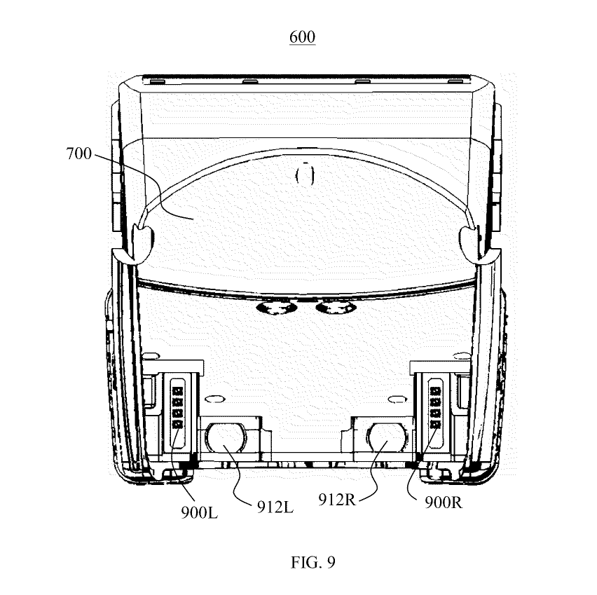

FIG. 2 shows an imaging system of device 100 in cross-section, according to one embodiment of the present invention. In this embodiment, device 100 comprises imaging system 200 including a sensor 201, objective lens 202, an alignment system 203, a reflecting device 205 (e.g., a fold mirror), a pin 207, and reflecting device 208 (e.g., a fold mirror). Although not present in the cross-section of FIG. 2, in one example device 100 contains an illumination source designed to illuminate the eyes of the subject and the resulting reflected light to be detected and imaged by imaging system 200. For example, an illumination source is depicted in FIGS. 8 and 9 in reference to the embodiment of device 600.

With reference to FIGS. 1A, 1B, 1C, and 2, in one example, bottom cover 104 is open allowing light reflected from an iris to enter into imaging system 200 of the iris capture device 100 provided it follows a path outlined by a speckled area 206. When iris capture device 100 is opened for capturing iris images, bottom cover 104 rotates about pin 207 into an open position. Additionally, or alternatively, pin 207 may be a hinge or any other type of attachment mechanism that permits the rotation described. In permitting this rotation, fold mirror 205 that is in position 210 (dashed rectangle) rotates in a direction denoted by arrow 209 into an open position denoted by a solid rectangle 205.

In one example, an optional alignment system 203 can be used to align the eyes of the subject. Although there are multiple options for the exact location of visible light alignment system 203, in the example shown alignment system 203 may be placed behind fold mirror 205 in its open position. When bottom cover 104 is in the closed position, alignment system 203 folds up inside of housing 108 of device 100.

In one example, fold mirror 205 is a dichroic mirror, such that it transmits visible light spectrum, for example light which may be used in alignment system 203, while reflecting near infrared light, e.g., light that may be used by imaging system 200 when imaging an iris onto sensor 201. As an alternative for fold mirror 205, a diffractive element such as a volume diffractive element made of dichromated gelatin or holographic photopolymer materials, can be used as a chromatic beam splitter. By way of example, the visible light of alignment system 203 can be in a green spectral region (e.g., about 500 nm to 550 nm) and produced by, e.g., a LED, while the light collected for the iris image has wavelengths in the 700 nm to 900 nm range, which are also capable of being generated by LEDs.

In various examples, illumination may be direct illumination provided by a light bulb, laser light, LED, or similar light source. Illumination may also be in the form of a diffuse illumination, whereby a means (not illustrated) of dispersing light is employed so that the source of illumination is not directly visible to a user of scanner 100. For example, a frosted piece of glass or plastic may be placed between a source of illumination and the eyes of the subject, ensuring that the light, which reaches the subject's eyes is diffused or softened and preferably homogenized to provide uniform illumination of the subject's eye(s). Other means to soften or diffuse the scanner illumination may be employed as well. This may make it possible to provide effective illumination for scanning the eyes of the subject, while not drawing the subject's line of sight directly to the source of illumination. It may be desired, for example, that the subject's line of sight is directed to the imaging optics, rather than the source of illumination.

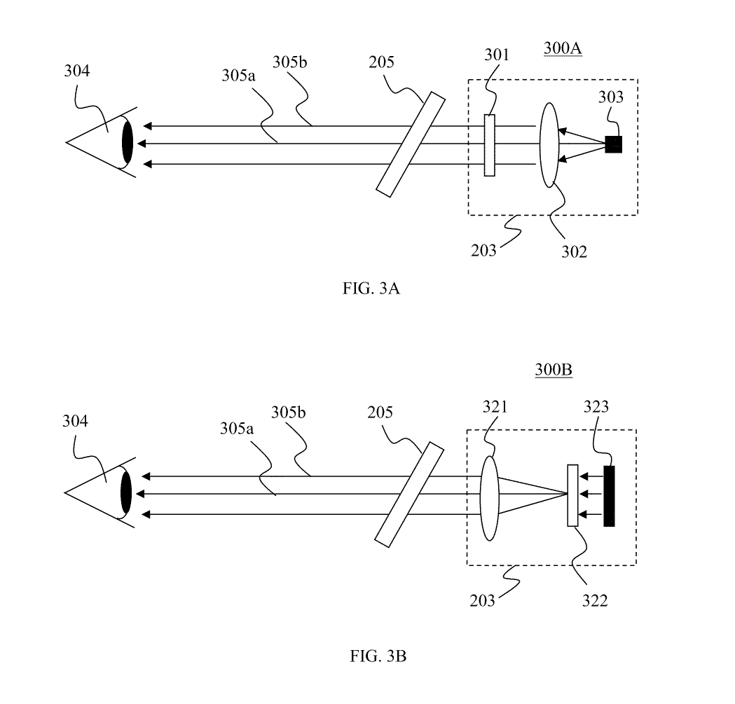

Alignment systems ensure alignment of the eyes of the subject with illumination from the scanner as well as imaging system 200 of the scanner. In particular, guidance is provided to the user as to the direction to which the subject should look such that the subject's iris(es) is within the FOV of imaging system 200 and the iris(es) are oriented substantially perpendicular to the imaging system's optical axis. FIG. 3A and FIG. 3B show various embodiments of alignment systems 300A and 300B. For example, these alignments systems 300A and 300B may be exemplary embodiments of alignment system 203.

FIG. 3A shows alignment system 300A comprising a light source 303 (e.g., a LED), an optical element 302 (e.g., a collimating lens composed of one or more optical elements), and a phase element 301. For example, phase element 301 may be designed to project a desired pattern in the far-field (e.g., at infinity or at a particularly long distance away from the phase element 301). In this arrangement, the alignment system 300A generates a set of collimated rays (rays 305a and 305b, for example) that have a phase imparted on them by phase element 301. Such phase plates (also termed pattern generators), by way of example, are produced and sold by Tessera (Charlotte, N.C.) and RPC Photonics (Rochester, N.Y.). An eye 304 of a subject focuses these rays onto its retina and perceives an object as if it were at infinity, or at least a long distance away. This object may appear as simply a dot or set of dots, a grid, crosshair, company logo, etc.

In a second embodiment, depicted in FIG. 3B, alignment system 300B comprises an illumination source 323, a patterning device 322 (e.g., a reticle), and an optical element 321 (e.g., a collimating element composed of one or more optical elements). Illumination source 323 can uniformly illuminate reticle 322 which may, by way of example, be a chromium (Cr) or ink-patterned glass or plastic substrate that represents a 2-D object such as a grid, series of points, company logo, figure of a person, etc. and may be acquired from a variety of companies including Max Levy Autograph (Philadelphia, Pa.) and Applied Image, Inc. (Rochester, N.Y.). As depicted by the parallel rays 305a and 305b generated by optical element 321, eye 304 of the subject will see an image of the reticule 322 at infinity. However, by adjusting optical element 321, the rays may need not be collimated, such that the image of the reticle 322 can appear located at any distance away from the subject's eyes 304, but desirably at a distance sufficiently large that the subject's eyes 304 can comfortably focus on it.

In one example, for both of the alignment systems 300A and 300B, the image content and/or position presented to the subject's eye may be different for the left and right eye. Also, in one example, the light from the alignment system 300A and 300B can be switched on for only one eye at a time, or may project two slightly different images to each eye so that a stereoscopic image is produced. In another example, for both of the alignment systems illustrated, fold mirror 205 may be a dichroic mirror, such that a visible light portion of beams generated by light sources 303 and 323 passes through the mirror 205, while the wavelength used (e.g., near-infrared) for the iris image capture is reflected. By way of example, light source 303 or 323 can be green light at about 550 nm, while iris illumination light beam can be about 760 nm infrared. In one example, fold mirror 205 can be fabricated with a dichroic coating, such that it reflects the near-infrared light and transmits the visible light.

In one example, to further suppress ambient light, it is the object of an embodiment of the present invention to utilize illumination wavelengths that are not present or minimally present in the spectrum of the ambient light. In the case of sunlight being the critical ambient light to suppress, it is an object of an embodiment of the present invention that iris illumination wavelengths that correspond to holes in the solar spectrum be utilized.

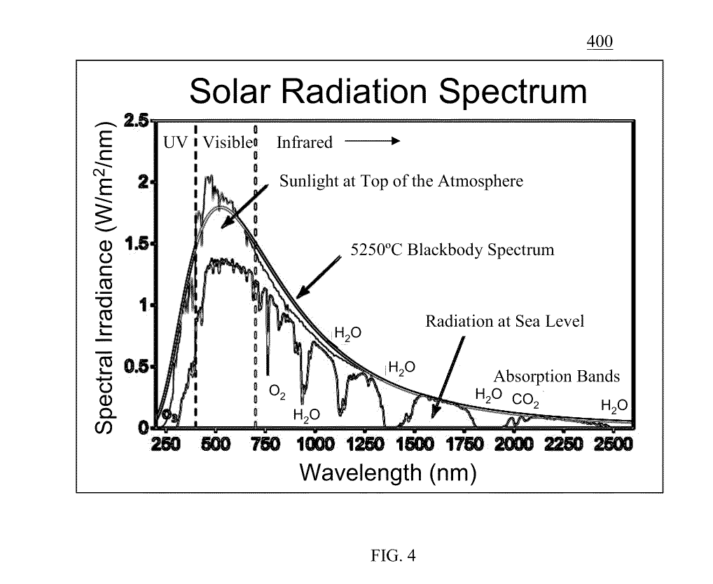

As illustrated in FIG. 4, a solar spectrum at sea level has several holes (or absorption bands) in the near infrared (IR) spectrum. There is an O₂ absorption line at about 760.5 nm that is about 2-3 nm wide, and there are several H₂O absorption lines in the about 900 nm, 1125 nm, 1400 nm and 1825 nm wavelength ranges. If an illumination source spectral width falls within the width of the hole of the solar spectrum, a spectral filter may still be required on the imaging side in order to filter out ambient light that is outside of this spectral hole. In one example, by implementing an illumination source at one or more of the solar spectral hole wavelengths and by using a spectral filter to filter out wavelengths outside of these spectral holes, the effect of ambient light can be dramatically reduced.

In one example, to further suppress ambient light, it is a further object of an embodiment of the present invention that the light illuminating the iris is polarized. The polarized light can be achieved using a source that is polarized, such as a laser or by incorporating an optical element, such as a sheet polarizer to polarize an otherwise randomly polarized optical light source, such as a LED. Further, a polarizing optical element is placed in the imaging optics path to act as a polarization analyzer for the light reflected off of the subject's face and iris. The two polarizers may be crossed or parallel to each other. However, by having the analyzing polarizer at the sensor perpendicular to the polarization of the illumination source, specular reflections off of the cornea of the eye can be minimized and primarily light that is depolarized such as that scattered off of the iris of the subject is imaged by the sensor.

In one example, a sensor of the type used in one or more of the above embodiments can have a spectral sensitivity range that is larger than a hole in the ambient light's spectrum. A spectral filter may operate as a narrowband pass filter or a narrowband reject filter depending upon whether or not the filter is used in transmission or reflection, respectively. Typically, however, the performance of a spectral filter will vary as a function of angle. With spectral filters fabricated using a series of dielectric coatings, the center wavelength will shift to shorter wavelengths as the incident angles increases according to:

λ(θ)=λ₀\sqrt{1−(sin θ/n_{eff})²}, (1)

where λ₀ is the center wavelength at normal incidence and n_{eff} is the effective index of refraction of the coating stack. For example, taking about η_{eff}=1.7567 (e.g., for U-grade notch filters from Semrock of Rochester, N.Y.), and assuming the center wavelength to be about λ₀=760.5 nm, a wavelength corresponding to an O₂ solar spectrum hole, changes in incident angle of +/−5.2° will create a 1 nm shift in the center wavelength. Given that absorption line is about 3 nm wide, it is critical that the range of angles incident upon the filter be minimized. By way of example, if the filter is placed between the imaging lens and the sensor, it is therefore preferable to have an imaging system that is telecentric in image space, such that the cone of rays hitting the sensor are the same angular orientation across the field of the sensor.

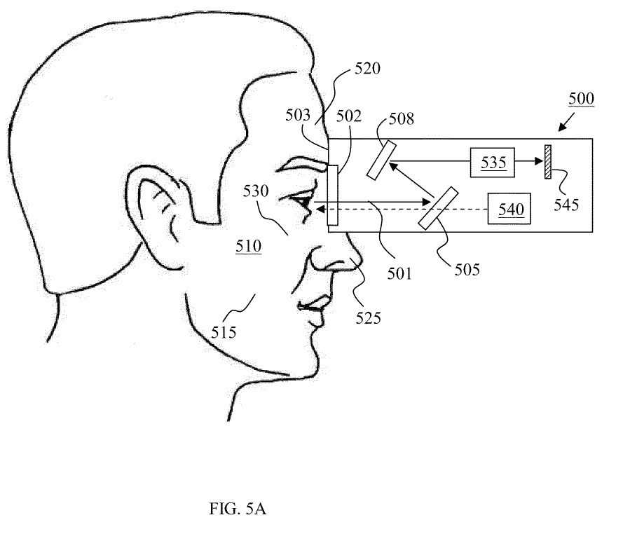

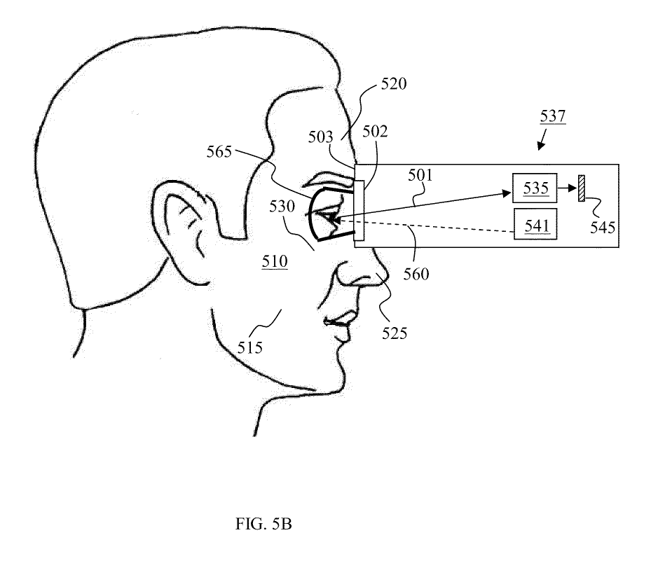

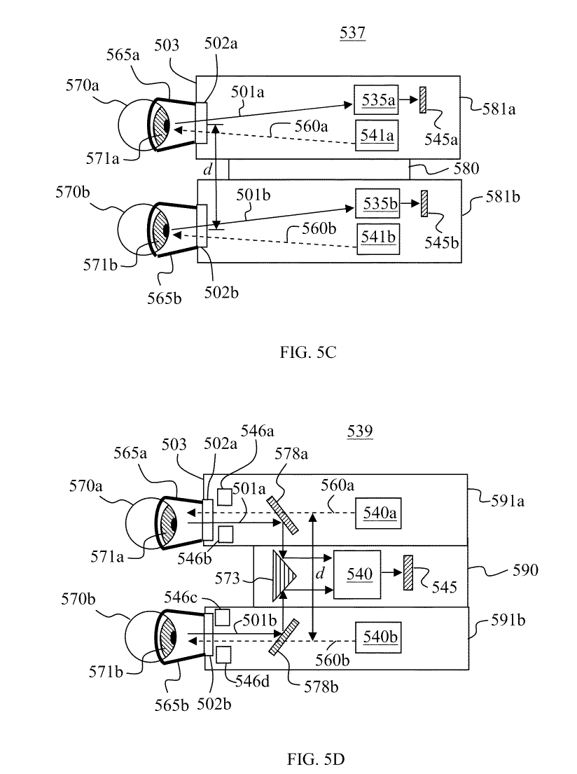

FIG. 5A shows an iris scanning device 500, according to an embodiment of the present invention. Device 500 does not open in order to capture an image of one or two irises of subject 510. In one example, device 500 provides a front housing 503 that is contoured to a face 515 of the subject, such that device 500 makes contact with the subject's face 515. Various embodiments of the contouring provide reproducible contact with the subject's face 515, such that the subject's eyes are the appropriate distance from the iris scanning device 500.

In one example, the contouring defines a plane on the head of the subject that is perpendicular or substantially perpendicular to the optical axis of the iris illumination and imaging system. One skilled in the art will appreciate that a wide variety of approaches to the contouring will meet this objective and are within the scope of embodiments of the present invention. For example, contouring can be designed to accommodate a three-point contact, a line contact, or two points and a line contact with the subject's face 515. The two points and a line approach define contact by way of a line across the forehead 520 of the subject, with the two points being, e.g. each cheek 530 of the subject's face. For example, front housing 503 can be contoured to conform to a brow 520, nose 525, or cheekbones 530 of the subject. In one example, the contour of front housing 503 shields the subject's iris or irises from ambient light in order to maximize the signal-to-noise ratio (SNR) of the captured iris image. In one example, light from an illumination system (not shown) within apparatus 500 is reflected off of an iris (for example, ray 501) and enters into apparatus 500 through a window 502. In the embodiment illustrated in FIG. 5A, the reflected iris light reflects off of a reflecting device 505 (e.g., a dichroic mirror), off a reflecting device 508 (e.g., a fold mirror), and is imaged onto a sensor 545 by an optical element 535. In this arrangement, by placing the apparatus against facial features of the subject, the distance between the subject's iris and the iris imaging system of the apparatus is set, thereby reducing the depth of focus requirements for the optical system.

In one example, alignment system 540 is shown providing a visible target that the eyes of the subject can focus upon. Alignment system 540 projects the visible target through to the eyes of the subject by way of the reflecting device 505, which for the frequency of the visible light used, acts to transmit the light through to the eyes of the subject. The reflecting device 505 can use a number of different approaches, including a dichroic plate, or a beam splitter. For example, a diffractive element such as a volume diffractive element made of dichromated gelatin or holographic photopolymer materials, can be used as a chromatic beam splitter.

In one example, window 502 is made of a material that is substantially transparent to the operating wavelengths of the iris illumination system and those of alignment system 540. The material, by way of example could be glass or plastic such as borosilicate glass, polycarbonate or acrylic. For applications using severe abrasion resistance, a window composed of sapphire may be considered or alternately a glass or plastic window with a hard coating added.

FIGS. 5B through 5D illustrate embodiments of the present invention where the apparatus does not have a single ambient light cover to shield both eyes (as in FIGS. 1A through 1C and FIG. 2), but rather has individual ambient light covers that shield a single eye or each of two eyes. FIG. 5B depicts a side view of apparatus 537. A cup 565 designed to contour around the eye is used to shield the eye from ambient light, such that it is only or primarily illuminated by light 560 emanating from light source 541. Cup 565 can be made of a soft material so as to be comfortable for the subject and so that by being at least partially compliant can achieve a more light-tight seal between the subject's eyes and the optical system of apparatus 537. Housing front 503 may or may not be used to set the distance of the apparatus from the subject's eyes in the presence of cups 565. If housing front is not used to set the distance, then cups 565 are preferably used to set the distance. Reflected light 501 from the eye is collected by imaging system 535 and the light imaged onto sensor 545. Note that as depicted, this embodiment does not include a fold mirror to fold the optical path of the imaging system or illumination system. A fold mirror can be added to apparatus 537 just as a fold mirror can be removed from apparatus 500 by redesigning the optical and illumination system.

Referring to FIG. 5C, apparatus 537 is depicted in a top-down view where two eyes 570a and 570b of a subject are imaged, for example to capture images of irises 571a and 571b. Alternatively, only one of the two eyes and irises may be captured by apparatus 537. In the two-eye capture embodiment depicted in FIG. 5C, two twin optical systems in housings 591a and 591b are utilized. In this embodiment, each uses a cup (565a and 565b) to align the optical system to the eye (570a and 570b) and utilizes a light source (541a and 541b) to illuminate the eye with rays 560a and 560b. Reflected light beams 501a and 501b are collected by imaging systems 535a and 535b and imaged onto sensors 545a and 545b. Mechanics 580 joining housings 581a and 581b allow the distance d to be adjusted in order to accommodate the interpupillary distance (IPD) of an individual subject. This adjustment can be conducted by the operator or the subject. This adjustment can be adjusted based upon signals from the apparatus regarding the location of the eyes analyzed in the captured image or based upon the subject adjusting the distance d (using a thumb wheel, slider mechanism or other means of adjusting the distance, not shown) until the subject can clearly see certain alignment aids (not drawn, but by example could incorporate those of FIGS. 3A and 3B) with both eyes.

Alternatively, the two-eye capture apparatus 537 may utilize a single sensor where the image of both irises 571a and 571b are imaged by a single optical system 540, as shown in FIG. 5D. The combining of two fields of view onto a single sensor can be accomplished via mirrors 578a, 578b, and 573, though this may also be achieved with prisms as well. Mirrors 578a and 578b can be dichroic, such that they transmit light (e.g., visible light) from alignment aids 540a and 540b, while reflecting light from the irises 501a and 501b. The reflected light can be near infrared, though may be visible light as well. Light reflecting off of irises 571a and 571b emanate from lights sources 546a, 546b, 546c, and 546d, and can uniformly illuminate the eye(s) of the subject to be imaged by apparatus 539. Mechanism 590 allows for adjustment of the distance d separating housings 591a and 591b to accommodate the different IPD of subjects as described when referring to FIG. 5C in the context of mechanism 580.

Alternatively, for the embodiments of FIGS. 5C and 5D, the adjustment of distance d is performed if the field of view of the optical system comprising the objective lens 540 (or 540a and 540b) in conjunction with sensor 545 (or 545a and 545b) allows for the capture of all subjects across a range of reasonably expected IPD (for example 52 to 72 mm). In this case, mechanisms 580 and 590 would not move parts for the adjustment of d, though they may still contain moving parts if the two housings 581a and 581b (or 591a and 591b) are designed to fold onto themselves or slide together in the interest of reduced volume and portability of apparatus 537 and 539 when not in use.

Additionally, or alternatively, exemplary iris scanner 100, 500, 537 or 539 may contain additional elements for accepting, focusing, and processing an image of at least part of an eye of a person, or for otherwise enabling scanner 100, 500, 537 or 539 to perform its intended functions. Such elements or components (not illustrated in any of the figures) may be contained within scanner housing 108 or may be behind lens or lenses or behind illumination and may include, for example, and without limitation:

power delivery and management components (including, for example and without limitation, batteries, transformers, power regulators, and similar components);

additional optical elements (including, for example and without limitation, lenses, prisms, mirrors, gratings, fiber optics, light-emitting elements, holographic components, and optical filters) for receiving, focusing, steering, filtering, and/or otherwise optically processing an image of the human eye;

image processing elements, including, for example and without limitation, charge-coupled devices (CCDs), complementary metal-oxide semiconductor (CMOS) active pixel sensors, amplifiers, digital-to-analog convertors (DACs), and analog-to-digital convertors (ADCs), for transforming an image of at least part of an eye of a person, received by the optical elements, to a signal suitable for image processing;

signal processing elements (including, for example and without limitation, a DSP, a microprocessor, and/or memory) for identifying from the obtained signal such physiological features as an iris of a human eye, a retina of the human eye, and possibly other features of the human eye;

information processing elements for identifying a person or persons based on the identified physiological features of a human eye; and

a transmitter or other means to relay information to an external data processing system.

Scanner 100, 500, 537 or 539 may also have additional external features, not illustrated, which enable scanner 100, 500, 537 or 539 receive power and/or to communicate data to and/or receive data from an external controller such as a personal computer. Such external features may include, for example and without limitation, one or more power connector(s), USB port(s), IEEE 1392 port(s), Ethernet port(s), infrared port(s), serial port(s), parallel port(s), RJ-11, RJ-14, RJ-25, and RJ-45 connector(s), other modular jack(s), and other ports, jacks, and connectors well known in the art. Additionally or alternatively, scanner 100, 500, 537 or 539 may incorporate wireless communication electronics that follow such protocols as IEEE 802.11.

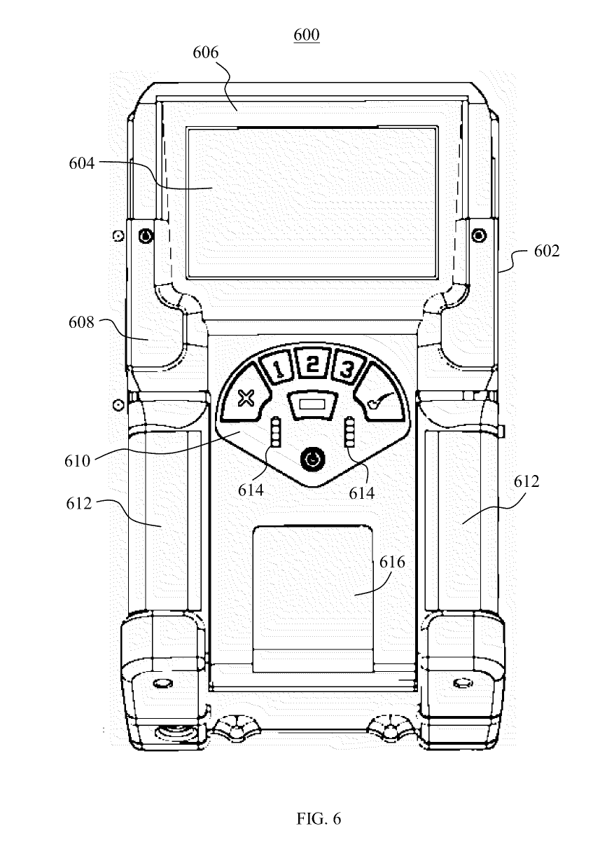

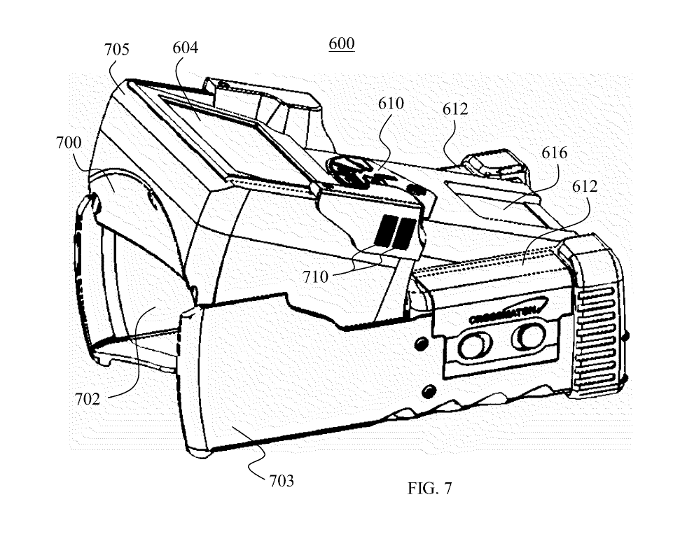

FIG. 6 depicts a top perspective view of an embodiment of a portable biometric device 600, e.g., in a closed non-operating position. Biometric device 600 comprises a housing 602, a display 604 occupying a first portion of a face 606 of an upper cover 608 of housing 602, a keypad 610 occupying a second portion of face 606 of upper cover 608 of the housing (the keypad provides for entry of data into the biometric device 600), one or more removable batteries 612 affixed to opposing sides of housing 602, one or more battery charge indicators 614 adjacent to keypad 610, and a fingerprint platen 616 occupying a second portion of face 606. The fingerprint platen may, by way of example, be part of a 2-finger capture device such as the Cross Match Technologies (Palm Beach Gardens, Fla.) V320 optical fingerprint scanner refolded or otherwise modified to fit within portably biometric device 600. Note that the keypad is drawn to indicate that the portable biometric device 600 can have a user-interface, but the sparse number of buttons contained by the keyboard as drawn are not be construed as a limitation of the depicted embodiment. The keyboard, by way of example, may include a sparse number of keys such as directional arrows and an execute button, or a numeric keypad, an alphabet keypad, a mouse pad, or any combination thereof. Additionally, or alternatively, user inputs may be accepted by the device 600 by making display 604 a touch screen (by way of example a capacitance or resistive touch screen) that accepts input from an operator's fingers, a pen directed by the operator, or any other pointing device. As will be apparent in FIGS. 8 and 9, an iris capture device may be integrated into device 600. To capture images of a subject's eyes the device is opened as depicted in FIG. 7.

FIG. 7 depicts a perspective view of device 600 in an open position wherein a first portion 705 of device 600 pivots or rotates upward and out of a second portion 703 of device 600. First and second portions 705 and 703, respectively, may be attached by an attachment mechanism at one end, such that rotation is permitted about that attachment mechanism. Suitable attachment mechanisms, as also described above, may include a pin, a hinge, etc. The perspective view of FIG. 7 illustrates an optional contoured area 700, which may receive a forehead (not shown) of a subject. The contoured area 700 may be located on first portion 705 of device 600 at an end of first portion 705 from where the portion interacts with the attachment mechanism. Also, an opening or cavity or area 702 of device 600 is shown, which is defined between first portion 705 and second portion 703. For example, in one embodiment, when contoured area 700 is in contact with the forehead of a subject (not shown), eyes (not shown) of the subject will be located proximate to opening 702. FIG. 7 illustrates a location of one or more optional USB connectors 710, which may be used for communication by biometric device 600 with the external environment. This perspective of device 600 also shows more details of a first battery 612.

FIG. 8 depicts more details of a left eye portion of the iris portion of optical system 600, according to one embodiment of the present invention. In this example, there are two light sources, one for each eye. For example, a left eye light source 800L is shown, as is left eye filter 812L. For example, left eye filter 812L may be used to remove ambient light from the reflected rays from the iris of the left eye.

FIG. 9 is a view of device 600 as the subject whose irises are to be imaged would see it, according to one embodiment of the present invention. In this embodiment, both eyes may be captured simultaneously or individually. Both the left and right eye light sources 900L and 900R are shown, as are left and right eye filters 912L and 912R. In one example, the depicted light sources are located to the side of the depicted filters, and behind the filters is the optical imaging system capable of imaging the reflected light off of a subject's eyes. The location of the light sources are drawn as examples only and are not considered to be limiting in their location since light sources may also be located as surrounding each of the left and right eye filter locations. Also shown in FIG. 9 is curvature 700 for receiving the forehead of the subject. The capture of both eyes simultaneously is advantageous because with simultaneous capture, one can determine head tilt from identifying certain predefined features in both the left and right iris image and connecting them. U.S. patent application Ser. No. 12/290,564 (filed Oct. 31, 2008), which is incorporated herein by reference in its entirety, discloses various example features available to the simultaneous capture approach. By way of example, the centroids of the identified pupils of each eye can be connected in order to determine the angle of the head tilt relative to the apparatus. Of course, simultaneous capture has consequences in terms of the power budget required to have both cameras working at once. In a further embodiment, an alternative is to use a larger sensor and a single optical system to capture both eyes in a single image frame of the sensor selected.

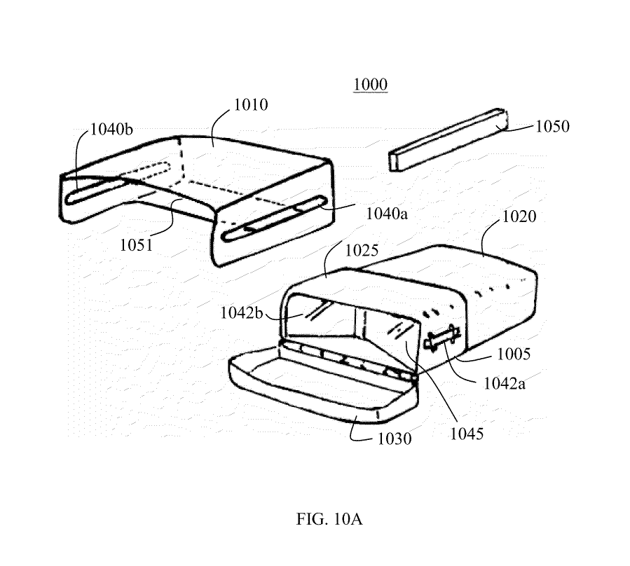

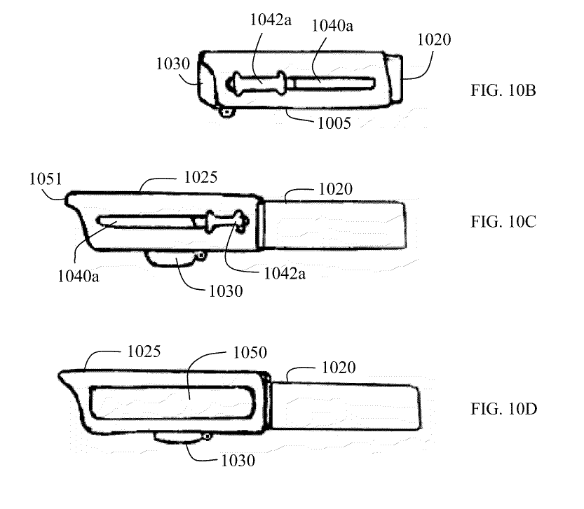

FIG. 10A is shows an exploded view of an iris scanner 1000, according to another embodiment of the present invention. Iris scanner 1000 includes a housing 1005, a hood 1010, a rear portion 1020, a body portion 1025, a front portion 1030, slots 1040a, 1040b on hood 1010, protrusions or extensions 1042a, 1042b on housing 1000, a window 1045, and an optional sealing device 1050.

In the example shown in FIGS. 10B, 10C, and 10D, an assembled view of iris scanner 1000 is shown, according to one embodiment of the present invention. In this example, housing 1005 is slidably coupled to hood 1010 through complementary coupling devices, e.g., slots 1040a, 1040b of hood 1010 and extensions/protrusion 1042a, 1042b of housing 1005. Other means of providing such functionality are within the scope of the present invention. FIG. 10B shows a collapsed view of the assembly, which FIGS. 10C and 10D show extended views of the assembly, as discussed in more detail below.

In one example, front portion 1030 may be an optical cover that is hingedly coupled to an edge of body portion 1025. In one embodiment, optical cover 1030 may be attached via a spring-loaded attachment mechanism.

In one example, a distance between the eyes of the subject and the iris scanning device 1000 are fixed by contact between a portion of the face of the subject and an edge 1051 of hood 1010. A shape of hood 1010 can be contoured to not only provide suitable contact points with the face of the subject, but also to block ambient light from above and below iris scanner 1000.

In one example, optional slot sealing attachments 1050 for hood 1010 can be provided to seal slots 1040a, 1040b while the iris scanning device is in operation, and thereby further block unwanted ambient light.

FIGS. 10B, 10C and 10D illustrate an embodiment of the iris scanner 1000 in various modes. FIG. 10B shows iris scanner housing 1005 in its closed or non-operational position within hood 1010. Optical cover 1030 is shown in its closed or up position. FIG. 10C shows iris scanner 1000 when hood 1010 is fully extended from or at a sliding stop position with respect to housing 1005 in its operating position, with optical cover 1030 in its extended or down position. FIG. 10D shows the use of slot sealing attachments 1050 to block the ambient light from entering through the sliding slots 1040.

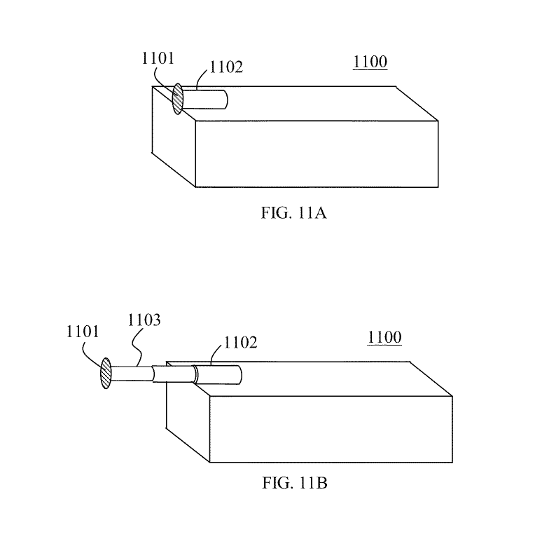

FIGS. 11A and 11B depict an iris capture apparatus 1100 that has a telescoping mechanical mechanism 1103 by which a distance from an iris of a subject iris and an apparatus' imaging system is maintained, according to an embodiment of the present invention. In FIG. 11A telescoping mechanism 1103 is in its closed position and contained within a cylinder 1102, while in FIG. 11B, telescoping mechanism 1103 is illustrated in its open position and fully extended with an end 1901 that is designed to touch a portion of a face of the subject, for example a center of a brow or forehead of the subject. As illustrated in this embodiment, no provisions are provided for ambient light rejection. Additionally, or alternatively, telescoping shielding may be added to the apparatus for such ambient light rejection. However, the iris capture apparatus may not require it due to the level of ambient light compared to the internal illumination source for illuminating the subject's iris. Alternatively, ambient light shielding may not be required if the apparatus operates at one or more wavelengths that correspond to a hole in the ambient light spectrum. In a further alternative, ambient light shields may not be required if spectral filters are used.

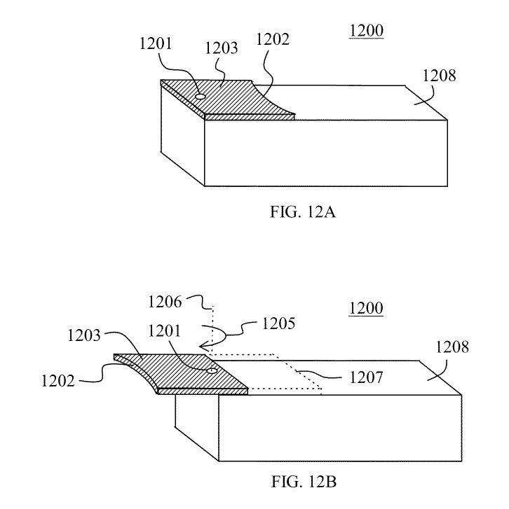

FIGS. 12A and 12B show an iris capture apparatus 1200 that has a mechanical plate 1203 situated on a top of a housing 1208, according to another embodiment of the present invention. In FIG. 12A mechanical plate 1203 is illustrated in its closed position. When the capture of an iris of a subject is desired, plate 1203 is rotated about an axis 1206 of pin 1201 in a direction indicated by arrow 1205 (see FIG. 12B), such that plate 1203 now extends out from iris capture device 1200. In one example, edge 1202 of plate 1203 is curved in order to accommodate the brow or forehead of the subject (not shown). The dashed lines 1207 illustrate the location of plate 1203 when in the closed position. Note that instead of rotation, plate 1203 can also slide or flip into position.

Though not illustrated, the embodiments of FIGS. 10 through 12 can have some form of locking or snapping mechanism whereby the open and closed positions of the sliding, telescoping, or rotating distant-setting mechanism has a default state of being open or closed, and not a state in between.

It is to be appreciated that, although only shown for one eye, either both eyes can be detected substantially simultaneously or each eye can be detected sequentially in time, as would be understood by a skilled artisan upon reading this description.

### Conclusion

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. An apparatus, comprising:
a housing; and
an iris capture device within the housing,
wherein the iris capture device is configured to capture an image of an iris of a subject, and
wherein a distance between the iris and the iris capture device is set according to a mechanical parameter of the housing.

2. The apparatus of claim 1, wherein a portion of the housing is configured to move in order to set the distance.

3. The apparatus of claim 1, wherein no portions of the housing move in order to set the distance.

4. The apparatus of claim 1, wherein the iris capture device is further configured to capture images of both irises of the subject.

5. The apparatus of claim 1, wherein the housing comprises a first portion that is coupled to a second portion of the housing via a rotating or slidably attachment mechanism.

6. The apparatus of claim 5, further comprising slot sealing attachments configured to block ambient light entering via the slidably attachment mechanism.

7. An apparatus, comprising:
a housing having a first cover and a second cover, wherein the first cover and the second cover are coupled through an attachment mechanism;
an illumination source coupled to at least one of the first cover and the second cover, wherein the illumination source is configured to illuminate an eye of a subject to produce reflected light; and
an imaging sensor coupled to at least one of the first cover and the second cover, wherein the imaging sensor is configured to receive the reflected light,
whereby an optical path is formed for the reflected light from the eye to the imaging sensor.

8. The apparatus of claim 7, wherein the attachment mechanism is configured to permit rotation of the second cover with respect to the first cover.

9. The apparatus of claim 8, wherein the attachment mechanism is a pin or a hinge.

10. The apparatus of claim 7, further comprising:
a window configured to permit illumination of the eye by the illumination source,
wherein the first cover and the second cover are configured to remain stationary with respect to each other.

11. The apparatus of claim 10, wherein the window comprises at least one of glass, plastic, borosilicate glass, polycarbonate, acrylic, and sapphire.

12. The apparatus of claim 10, wherein the housing serves as a mechanical stop against a face of the subject.

13. The apparatus of claim 7, wherein the optical path comprises:
a first mirror configured to redirect the reflected light from the eye into a volume adjacent to the first cover; and
a second mirror configured to further redirect the reflected light from the volume towards an end of the first cover closest to the attachment mechanism.

14. The apparatus of claim 7, wherein the housing serves as a mechanical stop against a face of the subject.

15. The apparatus of claim 14, wherein the mechanical stop is contoured to make one of three-point, a line and two-point, a line contact and a single point with a face of the subject.

16. The apparatus of claim 7, further comprising a mechanical bridge configured to make contact with a forehead of the subject.

17. The apparatus of claim 7, further comprising a recess in the second cover configured to make contact with a nose of the subject.

18. The apparatus of claim 7, wherein the second cover comprises a second shield with integrated side shields.

19. The apparatus of claim 18, wherein the second shield with integrated side shields is configured to slide into slots within the housing.

20. The apparatus of claim 19, wherein the slots contain gasketing-type material.

21. An apparatus, comprising:
a housing having a first cover and a second cover, wherein the first cover and the second cover are coupled through an attachment mechanism;
an illumination source coupled to at least one of the first cover and the second cover, wherein the illumination source is configured to illuminate an eye of a subject to produce reflected light; and
an imaging sensor coupled to at least one of the first cover and the second cover, wherein the imaging sensor is configured to receive the reflected light,
whereby an optical path is formed for the reflected light from the eye to the imaging sensor, and
wherein the illumination source produces a light beam having a frequency at approximately an ambient light minimum.

22. The apparatus of claim 21, wherein the frequency of the light beam is one of a water vapor absorption frequency and an oxygen absorption frequency.

23. The apparatus of claim 21, wherein the optical path further includes a spectral filter configured to pass the frequency and to block at least a portion of ambient light.

24. The apparatus of claim 21, wherein the housing serves as a mechanical stop against a face of the user.

25. The apparatus of claim 7, wherein the optical path includes a polarizer.

26. The apparatus of claim 7, wherein the illumination source is a light emitting diode or a laser.

27. The apparatus of claim 7, further comprising:
an alignment system including a target configured allowing the eye of the subject to focus.

28. The apparatus of claim 27, wherein the alignment system comprises one of a dichroic plate, a beam splitter, and a diffractive element.

29. The apparatus of claim 28, wherein the diffractive element comprises at least one of dichromated gelatin and holographic photopolymer material.

30. A system, comprising:
a first portion including a light capturing system and a portion configured to position a head of a subject for an iris measuring operation; and
a second portion including an illumination system that is configured to illuminate an iris of the subject and to direct reflected light from the iris onto the light capturing system.

31. A system, comprising:
a light source configured to illuminate an iris of a subject;
an optical system configured to capture light reflecting from the iris; and
a housing configured to hold the light source and the optical system, and configured to position a head of the subject to allow for proper light capture from the iris of the subject.

32. A system, comprising:
a first portion including a fingerprint capture system to capture one or more fingerprints or thumbprints of a subject and a surface configured to position a head of the subject for an iris capturing operation; and
a second portion including an iris capturing system configured to capture an image of the iris of the subject.

33. The system of claim 32, wherein the second portion comprises:
a light source;
an optical system; and
a detector.

34. The system of claim 33, further comprising left and right eye portions of the light source, the optical system, and the detector.

35. The system of claim 33, wherein the optical system comprises a lens and an ambient light filter.

36. A system, comprising:
a first portion including a first portion of an optical system and a surface configured to position a head of a subject to properly capture iris information from one or more eyes of the subject; and
a second portion including a second portion of the optical system,
wherein the first portion of the optical system includes a light source, optics, and an imaging device,
wherein the second portion of the optical system includes a reflecting device.

37. The system of claim 36, wherein:
a first light path from the light source to the iris allows for a reflection of a beam of radiation from the light source from the reflecting device in the second portion of the optical system onto the iris; and
a second light path from the iris to the imaging device allows for reflection of the beam of radiation, after reflecting from the iris, off of the reflecting device in the second portion of the optical system, off another reflecting device in the first portion of the optical system, through the optics, and onto the imaging device.

38. An apparatus, comprising:
a housing;
an iris capture device within the housing; and
a flexible flange,
wherein the iris capture device is configured to capture an image of a first iris of a subject,
wherein a distance between the first iris and the iris capture device is set according to a mechanical parameter of the housing, and
wherein the flexible flange is coupled to the housing, and is configured to limit ambient light from entering the iris capture device.

39. The apparatus of claim 38, further comprising:
another flexible flange coupled to the housing,
wherein the another flexible flange is configured to limit ambient light from entering the iris capture device, and
wherein a distance from the another flexible flange to the flexible flange is adjustable in response to an inter-pupillary distance of the subject.
